(19) United States
(12) Patent Application Publication
(10) Pub. No.: US 2007/0116702 A1
(43) Pub. Date: May 24, 2007

(54) Survival promoting NCAM binding and MCAM ligand binding compounds

(76) Inventors: Vladimir Berezin, Copenhagen (DK); Elisabeth Bock, Charlottenhund (DK); Lene Bording Kohler, Hvidovre (DK)

Correspondence Address: BROWDY AND NEIMARK, P.L.L.C., 624 Ninth Street, N.W., Washington, DC, 20001, US

(21) Appl. No.: 11/637,730

(22) Filed: Dec. 13, 2006

Related U.S. Application Data
(63) Continuation of application No. 10/450,150, filed on Nov. 24, 2003.

(51) Int. Cl. A61K 39/395; A61K 38/17

(52) U.S. Cl. 424/143.1; 514/12

(57) ABSTRACT

The present invention relates to compounds capable of stimulating survival of cells presenting the neural cell adhesion molecule (NCAM) or an NCAM-ligand (counter-receptor), such as neurons. Further, the present invention relates to the use of pharmaceutical compositions and medicaments in the treatment or protection of cells presenting NCAM or NCAM ligands. More particularly the invention describes the use of a compound comprising a peptide comprising at least 5 contiguous amino acid residues from an amino acid sequence of NCAM, a fragment thereof, or a variant thereof or a mimic thereof, for the preparation of a medicament for preventing cell death of cells presenting said NCAM or an NCAM ligand.

The present invention relates to compounds capable of stimulating survival of cells presenting the neural cell adhesion molecule (NCAM) or an NCAM-ligand (counter-receptor), such as neurons. Further, the present invention relates to the use of pharmaceutical compositions and medicaments in the treatment or protection of cells presenting NCAM or NCAM ligands.

### BACKGROUND OF THE INVENTION

Cell adhesion molecules (CAMs) constitute a group of proteins mediating adhesion between cells. A major group of CAMs belongs to the immunoglobulin (Ig) superfamily characterised by the presence of immunoglobulin domains. The neural cell adhesion molecule (NCAM) is such a cell adhesion molecule of the Ig superfamily that is particularly abundant in the nervous system. NCAM is expressed in the external membrane of nerve cells. When an NCAM molecule on one cell binds to another NCAM molecule on another cell (homophilic binding), the binding between the two cells is strengthened. NCAM not only binds to NCAM but also to other proteins and/or glycoconjugates found on nerve cells or in the extracellular matrix (heterophilic binding). NCAM also binds to ATP. By mediating adhesion between nerve cells—or between nerve cells and the extracellular matrix—NCAM influences migration of cells, extension of neurites, fasciculation of neurites and formation of synapses.

NCAM is encoded by a single gene, containing at least 25 exons. Due to alternative splicing of precursor mRNA, a variety of mature mRNA species and thereby protein isoforms of NCAM can be produced. Three major NCAM isoforms are generated by alternative splicing of exons 15 and 18 determining the mode of attachment of NCAM to the plasma membrane and the size of the intracellular NCAM domains, respectively. In the nervous system a glycosylphosphatidyl inositol (GPI) anchored 120 kDa isoform is expressed on the surface of glial cells, a transmembrane 140 kDa isoform is expressed on both neurons and glial cells, whereas a transmembrane 180 kDa isoform is found predominantly on the surface of neurons. The extracellular part of NCAM comprises five Ig-like homology modules (Ig1, g2, 1g3, 1g4 and Ig5) and two fibronectin type III modules (FnIII1 and FnIII2) (Berezin et al., 2000).

Heterophilic ligands of NCAM comprise a variety of heparan sulfate proteoglycans (e.g. agrin) and chondroitin sulfate proteoglycans (e.g. neurocan). NCAM Ig1 and Ig2 are probably the structural determinants of the interaction of NCAM with heparan sulfate proteoglycans since these two modules have been shown to bind heparin (Kiselyov et al. 1997). Reports on whether the core protein or the carbohydrate moieties are responsible for the binding of proteoglycans to NCAM are contradictory, and the contribution of this interaction to NCAM-mediated cellular functions is currently not understood (Retzler et al. 1996). The neural cell adhesion molecule L1 and the fibroblast growth factor (FGF) receptor are other heterophilic ligands of NCAM. The interaction between NCAM and L1 has been shown to be mediated by N-linked oligomannosidic glycans carried by L1 and a lectin-like binding site localised in the fourth Ig module of NCAM. Through this binding NCAM has been suggested to participate in a so-called assisted L1-L1 homophilic interaction (Horstkorte et al., 1993) presenting an interesting example of co-operation between two neural CAMs.

NCAM plays a crucial role during the development of the nervous system and of organs, such as kidney, liver, bowel, heart, gonads, pancreas, and muscles. In the mature nervous system NCAM is important for the plasticity of neuronal connections associated with regeneration, learning and memory. In the peripheral nervous system NCAM is involved in the initiation of outgrowth of nerve fibres and formation of nerve-muscle connections in regeneration after damage including lesions.

In signal transduction NCAM transduces extracellular signals leading to tyrosine phosphorylation and an increase in intracellular calcium concentration. Several models for the NCAM-NCAM binding have been proposed. It is suggested that cell adhesion and signal transduction is mediated through a reciprocal interaction between all five Ig domains, or at least between the Ig3 domains of two opposing NCAM molecules of the NCAM domains. One hypothesis predicts that homophilic NCAM adhesion is mediated by a trans-reciprocal interaction between the Ig3 domains of two opposing NCAM molecules. Recent structure studies indicate, however, that a double reciprocal interaction between Ig9 and Ig2 domains of two opposing NCAM molecules mediates homophilic NCAM binding (Thomsen et al., 1996; Kiselyov et al., 1997; Jensen et al., 1999; Kasper et al., 2000)

NCAM binding compounds capable of stimulating differentiation and/or neurite outgrowth from cells presenting NCAM are disclosed in WO 00/18801, in which the compounds are used in the treatment for regeneration of NCAM presenting cells.

The identification of one such compound, C3, is described by RØnn et al. (1999). C3 stimulates outgrowth by activating a signalling pathway identical to that activated by homophilic NCAM binding.

Various factors may cause neuronal cell death. Preventing neuronal cell death in individuals being exposed to risk factors causing cell death may be called maintaining/stimulating or promoting survival of the cells, or it may be called neuroprotection.

When neuronal cells are damaged, e.g. by reduced oxygen supply, the processes of cell death start and lead to cellular dysfunction, “collapse” of the intercellular communication between cells (network), retraction of cell processes and eventually cell death. Preventing neuronal cell death, i.e. stimulating/promoting survival means that the cells are protected from initiation of the processes of cell death.

Survival has been discussed in some references, for example Hulley et al. (1998) disclose that the L1 neural cell adhesion molecule is capable of stimulating survival and differentiation in fetal mid-brain dopaminergic neurons cultured in the presence of the toxin MPP+.

U.S. Pat. No. 6,037,320 describes the identification of a neurotrophic factor, NT-4 and in U.S. Pat. No. 5,767,240 an activity-dependent neurotrophic factor capable of increasing the survival of spinal cord neuronal cells, cerebral cortical cells and hippocampal neurons is revealed.

Further, U.S. Pat. No. 5,567,682 concerns a method of treating the symptoms of Alzheimer's disease by intranasal administration of short chain peptides. The peptides promote neuronal survival by reducing or halting progressive neuronal degeneration.

The inventors of the present invention have surprisingly found that synthetic ligands of NCAM, e.g. C3, and peptide sequences from the neural cell adhesion molecule are capable of preventing cell death of cells presenting NCAM or NCAM ligands (i.e. counterreceptors).

### SUMMARY

The present invention relates to the use of a compound comprising a peptide sequence comprising at least 5 contiguous amino acids from the neural cell adhesion molecule (NCAM) or a variant thereof or a mimic thereof, for the preparation of a medicament for preventing cell death of cells presenting said NCAM or an NCAM ligand or a mimic thereof.

The present invention also provides for using the medicament in the treatment of diseases or conditions of the nervous system, the muscles and heart.

### FIGURES

FIG. 1: shows the effect of the C3d peptide on survival of PC12 cells after withdrawal of NGF.

FIG. 2: shows the effect of the peptide C3d on survival of PC12 cells in the absence of growth factors. The d in C3d refers to the fact that the C3 peptide can be synthetised as a tetrameric dendrimer.

FIG. 3: shows the effect of the C3d peptide on survival of cerebellar granule cells.

FIG. 4: shows the effect of the C3d peptide on survival of dopaminergic neuron.

FIG. 5: shows the effect of the FGL peptide on survival of granule cells.

FIG. 6: shows the effect of the monomeric FGL peptide on survival of granule cells.

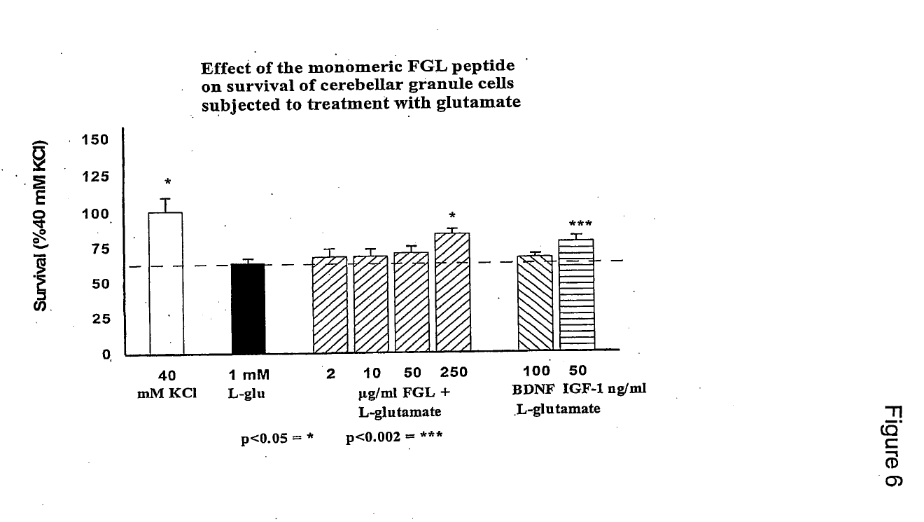

FIG. 7: A and B: show different forms of NCAM, A) the main forms of NCAM all have similar extracellular parts consisting of five immunoglobulin-domains (Ig-domains) and two Fibronectin type III-domains (FnIII-domains). Three transmembrane or membrane attached forms (NCAM-120, -140 and -180) are generated by alternative splicing. In addition, various soluble NCAM forms (NCAMs) exist, B) individual NCAM-domains are numbered from the N-terminal (NH₂), the most N-terminal domain being termed NCAM Ig1. An important alternatively spliced exon is the VASE exon that can be inserted in the region encoding the Ig4 domain of NCAM. The Ig5 domain can be glycosylated with polysialic acid (PSA).

FIG. 8 shows an identification of bead-coupled peptides binding NCAM do mains, A) libraries of bead-coupled peptides are incubated with the recombinant NCAM Ig1 domain. Beads that bind e.g. NCAM Ig1 are visualised by a staining reaction. Stained beads are isolated and micro-sequenced (Example 3 and 4), B) after evaluation of binding sequences, peptides corresponding to these sequences are synthesised as monomers, dendrimers (4-mers) or BSA-coupled 20-mers (Example 5), C) structure of peptide dendrimers. Four peptide-monomers (“peptide”) are coupled to a backbone consisting of three lysines.

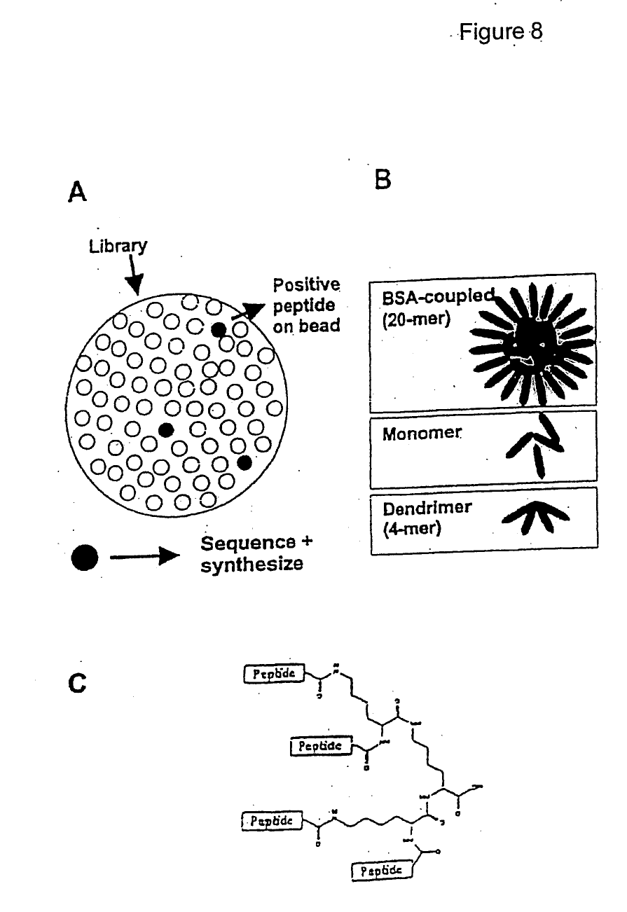

### DETAILED DESCRIPTION OF THE INVENTION

The compounds according to the invention relate to the prevention of neuronal cell death. Peripheral nerve cells possess to a limited extent a potential to regenerate and re-establish functional connections with their targets after various injuries. However, functional recovery is rarely complete and peripheral nerve cell damage remains a considerable problem. In the central nervous system, the potential for regeneration is even more limited. Therefore, the identification of substances with the ability to prevent neuronal cell death in the peripheral and the central nervous system is of great interest.

In the present context the wording “stimulate/promote survival” is used synonymously with the wording “preventing cell death” or “neuroprotection”. By stimulating/promoting survival it is possible to prevent diseases or prevent further degeneration of the nervous system in individuals suffering from a degenerative disorder. “Survival” refers to the process, wherein a cell has been traumatised and would under normal circumstances, with a high probability die, if not a compound of the invention was used to prevent said cell from degenerating, and thus promoting or stimulating survival of said traumatised cell.

The present invention relates to the use of compounds comprising a peptide sequence of at least 5 contiguous amino acids from the neural cell adhesion molecule (NCAM), a fragment thereof or a variant thereof or a mimic thereof, for the preparation of a medicament for preventing cell death of cells presenting said NCAM or an NCAM ligand.

By the term “cells presenting NCAM” is meant cells expressing SCAM in the external membrane of the cells, these cells are for example neurons, glial cells, all types of muscle cells, neuroendocrine cells, gonadal cells and kidney cells.

By the term “cells presenting an NCAM ligand” is meant cells expressing a receptor or ligand whereto NCAM and/or parts of NCAM may bind (i.e.: socalled counterreceptor). Examples of NCAM ligands are the FGF (fibroblast growth factor) receptor, L1 or proteoglycans, such as heparin, heparan sulphateproteoglycans, and chondroitin sulphateproteoglycans.

In particular the invention relates to the use of a compound wherein at least 5 contiguous amino acid residues are selected from the amino acid sequence of Ig9, Ig2, Ig3, Ig4, Ig5 of NCAM, or a fragment thereof, or a variant, or a mimic thereof.

The “fragment thereof” is to be understood as being any part of the NCAM molecule capable of binding to NCAM or an NCAM ligand/receptor and through said binding prevent cell death of the cell presenting NCAM or the NCAM ligand. The “variant thereof” is to be understood as being any peptide sequence capable of binding to NCAM or NCAM ligand/receptors, and via said binding preventing cell death of the cell presenting NCAM or the NCAM ligand. Thus, fragment or variant may be defined as
i) Fragments/variants comprising an amino acid sequence capable of being recognised by an antibody also capable of recognising the predetermined NCAM amino acid sequence, and/or
ii) Fragments/variants comprising an amino acid sequence capable of binding to a receptor moiety also capable of binding the predetermined NCAM amino acid sequence, and/or
iii) Fragments/variants having at least a substantially similar binding affinity to at least one NCAM molecule and/or NCAM ligand as said predetermined NCAM amino acid sequence

In the present context the term “functional equivalent” means a variant as defined above.

The binding affinity of the compound according to the invention preferably has a binding affinity (Kd value) to NCAM and/or the ligand in the range of 10⁻⁴ to 10⁻¹⁰ M, such as preferably in the range of 10⁻⁴ to 10⁻⁸ M. According to the present invention the binding affinity is determined by one of the following assays of surface plasmon resonance analysis or nuclear magnetic resonance spectroscopy.

In one embodiment the present invention relates to the NCAM Ig2 domain stimulating the survival of NCAM presenting cells. Thus, NCAM Ig2 is a ligand of the NCAM Ig1 domain. Further, the NCAM Ig2 domain stimulates survival by activation of NCAM specific signal transduction pathways.

Likewise, the present invention discloses the NCAM Ig1 domain as a ligand of the NCAM Ig2 domain being capable of stimulation of the survival of NCAM presenting cells by activation of specific signal transduction pathways.

The inventors have also, by means of combinatorial chemistry, identified small NCAM binding peptides which stimulate survival. Active peptides selected from a peptide library have been identified, and a putative motif comprising two or more basic amino acid residues has been identified. The peptides have been shown to stimulate the same specific signal transduction pathways as the NCAM Ig2 domain.

The results show that ligands of NCAM Ig1 or Ig2 domains or functional mimics hereof, which are capable of activating specific signalling pathways, can prevent cell death. Other functional mimics of the NCAM Ig1 or Ig2 domains, such as antibodies and non-peptide molecules may be beneficial in the same way. Therefore, the present invention provides compounds and compositions which are, or comprise small peptides, polypeptides, antibodies and non-peptide molecules recognising the NCAM Ig1 or Ig2 domains. When applied to tissue containing NCAM-expressing cells these compounds and compositions will promote NCAM function. The compounds and the compositions can be applied to promote survival of cells in the nervous system, the muscles and any other NCAM-expressing tissues, including various organs.

By means of nuclear magnetic resonance (NMR), the NCAM Ig2 domain was shown to belong to the I-set of Ig-domains (Jensen et al., 1999) as does the NCAM Ig1 domain that may be capable of binding to the NCAM Ig2 domain. By analysing the chemical shifts of the individual amino acid residues a distinct interaction site between the Ig1 and the Ig2 domain was found. It is thus parts of these two domains, which together form one distinct interaction/binding site for the NCAM binding compounds of the invention.

In the Ig2 domain, the amino acid frame Glu-190 to Phe-201 are particularly important for the binding according to the chemical shift studies and the indicated model. Similarly, in the Ig1 domain, the amino acids Glu-29, Glu-30, Glu-34, Glu-35 and Lys-37, Phe-38 and Phe-39 appear to be particularly important for the binding.

Without being bound by any particular theory, the inventors believe that active ligands of the NCAM Ig1 and/or the NCAM Ig2 domains are ligands which bind to the NCAM Ig1 domain and/or the NCAM Ig2 domain and thus trigger a conformational change of the domain resulting in a signalling cascade being initiated. This signalling influences differentiation and/or survival of cells. Thus, a suitable ligand may be any compound which can trigger a conformational change of the NCAM Ig1 domain and/or the NCAM Ig2 domain, resulting in a downstream messenger cascade.

The invention thus includes compounds which bind to either the NCAM Ig1 domain or the NCAM Ig2 domain. Together these two domains form the herein disclosed homophilic binding site.

In the present context, a mimic of the NCAM Ig1 and/or Ig2 domains should be understood to be any compound which binds to the NCAM Ig1 domain or the Ig2 domain, and through said binding stimulates survival of and/or differentiation of NCAM presenting cells, i.e. functional mimics. Mimics may be peptides, peptide derivatives, antibodies and non-peptide compounds such as small organic compounds, sugars and fats, as well as peptido-mimetics.

The invention also concerns non-peptide mimics of the NCAM Ig1 or Ig2 domains or the peptides defined above. In particular, such mimics should be understood to be compounds which bind to or in other ways interact with the NCAM Ig1 domain and/or the NCAM Ig2 domain and thereby stimulate survival from and/or differentiation of NCAM presenting cells.

In one embodiment mimics may be understood to exhibit amino acid sequences gradually differing from the preferred predetermined sequence, as the number and scope of insertions, deletions and substitutions including conservative substitutions increase. This difference is measured as a reduction in homology between the predetermined sequence and the mimic.

The peptides may be modified, for example by substitution of one or more of the amino acid residues. Both L-amino acids and D-amino acids may be used. Other modification may comprise derivatives such as esters, sugars, etc. Examples are methyl and acetyl esters. Polymerisation such as repetitive sequences or attachment to various carriers well-known in the art, e.g. lysine backbones or protein moieties such as bovine serum albumin (BSA) is also an aspect of the invention.

Mimics of the fragments according to the invention may comprise, within the same mimic, or fragments thereof or among different mimics, or fragments thereof, at least one substitution, such as a plurality of substitutions introduced independently of one another. Mimics of the complex, or fragments thereof may thus comprise conservative substitutions independently of one another, wherein at least one glycine (Gly) of said mimic, or fragments thereof is substituted with an amino acid selected from the group of amino acids consisting of Ala, Val, Leu, and IIe, and independently thereof, mimics, or fragments thereof, wherein at least one alanine (Ala) of said mimics, or fragments thereof is substituted with an amino acid selected from the group of amino acids consisting of Gly, Val, Leu, and lie, and independently thereof, mimics, or fragments thereof, wherein at least one valine (Val) of said mimic, or fragments thereof is substituted with an amino acid selected from the group of amino acids consisting of Gly, Ala, Leu, and lie, and independently thereof, mimics, or fragments thereof, wherein at least one leucine (Leu) of said mimic, or fragments thereof is substituted with an amino acid selected from the group of amino acids consisting of Gly, Ala, Val, and Ile, and independently thereof, mimics, or fragments thereof, wherein at least one isoleucine (Ile) of said mimics, or fragments thereof is substituted with an amino acid selected from the group of amino acids consisting of Gly, Ala, Val and Leu, and independently thereof, mimics, or fragments thereof wherein at least one aspartic acids (Asp) of said mimic, or fragments thereof is substituted with an amino acid selected from the group of amino acids consisting of Glu, Asn, and Gln, and independently thereof, mimics, or fragments thereof, wherein at least one aspargine (Asn) of said mimics, or fragments thereof is substituted with an amino add selected from the group of amino acids consisting of Asp, Glu, and Gln, and independently thereof, mimics, or fragments thereof, wherein at least one glutamine (Gln) of said mimics, or fragments thereof is substituted with an amino acid selected from the group of amino acids consisting of Asp, Glu, and Asn, and wherein at least one phenylalanine (Phe) of said mimics, or fragments thereof is substituted with an amino acid selected from the group of amino acids consisting of Tyr, Trp, His, Pro, and preferably selected from the group of amino acids consisting of Tyr and Trp, and independently thereof, mimics, or fragments thereof, wherein at least one tyrosine (Tyr) of said mimics, or fragments thereof is substituted with an amino acid selected from the group of amino acids consisting of Phe, Trp, His, Pro, preferably an amino acid selected from the group of amino acids consisting of Phe and Trp, and independently thereof, mimics, or fragments thereof, wherein at least one arginine (Arg) of said fragment is substituted with an amino acid selected from the group of amino acids consisting of Lys and His, and independently thereof, mimics, or fragments thereof, wherein at least one lysine (Lys) of said mimics, or fragments thereof is substituted with an amino acid selected from the group of amino acids consisting of Arg and His, and independently thereof, mimics, or fragments thereof, and independently thereof, mimics, or fragments thereof, and wherein at least one proline (Pro) of said mimics, or fragments thereof is substituted with an amino acid selected from the group of amino acids consisting of Phe, Tyr, Trp, and His, and independently thereof, mimics, or fragments thereof, wherein at least one cysteine (Cys) of said mimics, or fragments thereof is substituted with an amino acid selected from the group of amino acids consisting of Asp, Glu, Lys, Arg, His, Asn, Gin, Ser, Thr, and Tyr.

It is clear from the above outline that the same equivalent or fragment thereof may comprise more than one conservative amino acid substitution from more than one group of conservative amino acids as defined herein above.

Conservative substitutions may be introduced in any position of a preferred predetermined polypeptide of the invention or fragment thereof. It may however also be desirable to introduce non-conservative substitutions, particularly, but not limited to, a non-conservative substitution in any one or more positions.

A non-conservative substitution leading to the formation of a functionally equivalent fragment of the peptide of the invention would for example differ substantially in polarity, for example a residue with a non-polar side chain (Ala, Leu, Pro, Trp, Val, Ile,. Leu, Phe or Met) substituted for a residue with a polar side chain such as Gly, Ser, Thr, Cys, Tyr, Asn, or Gin or a charged amino acid such as Asp, Glu, Arg, or Lys, or substituting a charged or a polar residue for a non-polar one; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

Substitution of amino acids may in one embodiment be made based upon their hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include: arginine and lysine; glutamate and aspartate; serine and threonine; glutamine and asparagine; and valine, leucine and isoleucine.

The addition or deletion of an amino acid may be an addition or deletion of from 2 to preferably 10 amino acids, such as from 2 to 8 amino acids, for example from 2 to 6 amino acids, such as from 2 to 4 amino acids. However, additions of more than 10 amino acids, such as additions from 2 to 10 amino acids, are also comprised within the present invention. In the multimeric forms additions/deletions may be made individually in each monomer of the multimer.

It will thus be understood that the invention concerns compounds comprising at least one fragment capable of binding at least one receptor or a variant thereof, including any variants and functional equivalents of such at least one fragment.

A fragment comprising the Ig1 or Ig2 binding region of NCAM molecules is particularly preferred. However, the invention is not limited to fragments comprising the NCAM binding region. Deletions of such fragments generating functionally equivalent fragments comprising less than the NCAM binding region are also comprised within the present invention. Functionally equivalent peptides and fragments thereof according to the present invention, may comprise less or more amino acid residues than the NCAM binding region or regions of Ig1 and Ig2 capable of binding NCAM-ligands.

All functional equivalents of NCAM peptides are included within the scope of this invention, regardless of the degree of homology that they show to a predetermined sequence of the NCAM peptide or NCAM-ligand binding regions. The reason for this is that some parts of the binding regions are most likely readily mutatable, or capable of being peptide deleted, without any significant effect on the binding activity of the resulting fragment.

A functional equivalent obtained by substitution may well exhibit some form or degree of native NCAM activity, and yet be less homologous, if residues containing functionally similar amino acid side chains are substituted. Functionally similar in the present respect refers to dominant characteristics of the side chains such as hydrophobic, basic, neutral or acidic, or the presence or absence of steric bulk. Accordingly, in one embodiment of the invention, the degree of identity between i) a given functional equivalent capable of effect and ii) a preferred predetermined fragment, is not a principal measure of the fragment as a variant or functional equivalent of a preferred predetermined peptide fragment according to the present invention.

Fragments sharing at least some homology with a preferred predetermined fragment of at least 3 amino acids, more preferably at least 5 amino acids, are to be considered as falling within the scope of the present invention when they are at least about 25 percent homologous with the preferred predetermined NCAM peptide, or fragment thereof, or a mimic thereof, such as at least about 30 percent homologous, for example at least about 40 percent homologous, such as at least about 50 percent. homologous, for example at least about 55 percent homologous, such as at least about 60 percent homologous, for example at least about 65 percent homologous, such as at least about 70 percent homologous, such as at least about 75 percent homologous, for example at least about 65 percent homologous, such as at least about 80 percent homologous.

Sequence identity can be measured using sequence analysis software (for example, the Sequence Analysis Software Package of the Genetics Computer Group, University of Wisconsin Biotechnology Center, 1710 University Avenue, Madison, Wis. 53705), with the default parameters as specified therein.

In one embodiment the invention concerns the use of a compound capable of binding to the homophilic Ig1 binding site of the Ig1-Ig2 domains. The inventors have identified a homophilic binding site of NCAM which the NCAM domains Ig1 and Ig2 both contribute to. By “homophilic” binding site is meant that the site mediates binding between two identical molecules, in casu NCAM to NCAM. Thus, the invention relates to the use of a compound capable of binding to the NCAM Ig1 domain, such as Ig2, or a fragment, or a variant, or mimics thereof. Such compounds may be a peptide constituting the NCAM Ig2 domain, a fragment thereof or a mimic thereof, wherein fragment and mimic are as defined above.

The compounds may bind to the Ig2 binding site on the NCAM Ig1 domain or to a binding site different from the NCAM Ig2 binding site. It is believed that the ligands C3, D3 and D4 (described by RØnn et al., 1999) bind to a site different from the binding site of NCAM Ig2 or fragments thereof. Ig1 and Ig2 are constituted by the amino acid residues Val-18 to Val-210 of the amino acid sequence of NCAM. Ig2 alone is constituted by the amino acids Lys-121 to Val-210.

Such compound may be capable of binding to the NCAM Ig1 domain through a binding motif which comprises at least 2 basic amino acid residues. In one embodiment of the invention the binding motif comprises at least 2 basic amino acid residues, and at least 1 apolar amino acid.

In a further embodiment the at least 2 basic amino acid residues are within a sequence of 10 amino acid residues. In yet another embodiment the at least 2 basic amino acid residues are within a sequence of 3 amino acid residues.

A motif for binding to NCAM Ig1 has been identified. The motif (C3) includes positively charged amino acids in a relatively loose sequence-order, K/R (aa)₀₋₈ K/R, preferably K/R (aa)₀₋₁ K/R, wherein K and R designate lysine and arginine respectively, and the positively charged amino acids are separated by up to 8 amino acid (aa) residues. Preferably, however, the positively charged amino acids are adjacent or separated by only one amino acid residue.

Analysis of the active peptides isolated from the peptide library suggests that the motif may comprise more than two positively charged amino acids, for example three or four basic amino acids.

Preferred peptides comprise the sequence:
(Xaa⁺)ₘ-(Xaa)ₚ-(Xaa⁺)-(Xaa¹)ᵣ-(Xaa⁺)-(Xaa)_{q}(Xaa⁺)ₙ,
wherein Xaa⁺ is a basic amino acid residue,
Xaa¹ is any amino acid residue,
Xaa is any amino acid residue, and
m, n, p, q and r independently are 0 or 1.
   and wherein the basic amino acid residues preferably are lysine or arginine and r preferably is 1.

The nature of the amino acid residues Xaa and Xaa¹ does not seem to be important. It appears that they may be any amino acid residue. However, Xaa¹ is preferably proline (P) or glutamic acid (E).

In even more preferred peptides r is 1 and at least one of m and n is 1.

Preferred peptides of the invention comprise the sequence (K/R)₀₋₁-K/R-X-K/R), wherein X has the meaning of Xaa¹, suitably the sequence K/R-K/R-X-K/R or K/R-X-K/R, more suitably the sequence K/R-P-K/R, K/R-K/R-P-K/R, K/R-K/R-E-K/R or K/R-K/R-E-K/R and most suitably K-P-K, K-K-P-K, K-K-E-K or K-K-E-R. Examples are the sequences A-S-K-K-P-K-R-N-I-K-A (SEQ ID NO:1), A-K-K-E-R-Q-R-K-D-T-Q (SEQ ID NO:2), and A-R-A-L-N-W-G-A-K-P-K (SEQ ID NO:3).

According to the invention, peptides comprising the above sequence may be a part (hereinafter called a fragment) of the NCAM Ig2 domain or a mimic of the NCAM Ig2 domain. Furthermore, the peptides may bind to the Ig2 binding site of the Ig1 domain or to a different binding site on the Ig1 domain. If the binding site is not the “normal” Ig2 binding site, the binding will mimic the normal binding and result in survival of NCAM presenting cells in the same way.

Thus, the compound may comprise anyone or more of the following 22 sequences:

| | |
|---|---|
| ASKKPKRNIKA, | (SEQ ID NO:1) |
| AKKERQRKDTQ, | (SEQ ID NO:2) |
| ARALNWGAKPK, | (SEQ ID NO:3) |
| AGSAVKLKKKA, | (SEQ ID NO:4) |
| AKYVLIPIRIS, | (SEQ ID NO:5) |
| ASTKRSMQGI, | (SEQ ID NO:6) |
| ARRAILM(Q/T/N)-AL, | (SEQ ID NO:7) |
| AYYLIVRVNRI, | (SEQ ID NO:8) |
| ATNKKTGRRPR, | (SEQ ID NO:9) |
| AKRNGPLINRI, | (SEQ ID NO:10) |
| AKRSVQKLDGQ, | (SEQ ID NO:11) |
| ARQKTMKPRRS, | (SEQ ID NO:12) |
| AGDYNPDLDR, | (SEQ ID NO:13) |
| ARKTRERKSKD, | (SEQ ID NO:14) |
| ASQAKRRKGPR, | (SEQ ID NO:15) |
| APKLDRMLTKK, | (SEQ ID NO:16) |
| AKKEKPNKPND, | (SEQ ID NO:17) |
| AQMGRQSIDRN, | (SEQ ID NO:18) |
| AEGGKKKKMRA, | (SEQ ID NO:19) |
| AKKKEQKQRNA, | (SEQ ID NO:20) |
| AKSRKGNSSLM, | (SEQ ID NO:21) |
| ARKSRDMTAIK. | (SEQ ID NO:22) |

In particular the following three peptides, C3 (SEQ ID NO 1), D3 (SEQ ID NO:2) and D4 (SEQ ID NO:3) are preferred.

In yet another embodiment the invention relates to compounds, which may be peptides which bind to that part of the homophilic binding site of NCAM Ig1-Ig2 which is constituted by the Ig1 domain. Such peptides appear to have the general sequence, including any functional derivative thereof, as follows:
(Xaa)_{q}(Xaa⁺)-(Xaa)-(Xaa)-(Xaa)ₘ-(Xaa⁺)-(Xaa)-(Xaa⁻)ₙ-(Xaa^{h})-(Xaa)ₒ-(Xaa^{h})ₚ(Xaa⁺),
wherein Xaa⁺ is a basic amino acid residue,
Xaa⁻ is a an acidic amino acid residue,
Xaa^{h} is a apolar amino acid residue,
Xaa is any amino acid residue, and
m, n, o, p and q independently are 0 or 1,
   and wherein the basic amino acid residues preferably are lysine or arginine, the acidic amino acids preferably are glutamic acid or aspartic acid, the apolar amino acids are preferably leucine, isoleucine, valine or phenylalanine, and r preferably is 1.

Such a peptide may comprise the sequence (K/R)-X-X-X-(K/R)-X-(E/D)-(L/I/V/F)-X-(L/I/V/F), wherein X is any amino acid residue, suitably the sequence (K/R)X-(E/D)-(L/I/V/F)-X-(L/I/V/F), (K/R)-X-X-X-(K/R)-X-(E/D), (K/R)-X-X-(K/R)-X-(E/D) or (K/R)-X-(L/I/V/F)-X-(L/I/V/F), more suitably the sequences (K/R)-X-X-X-(K/R)-X-(E/D)-(L/I/V/F), (K/R)-X-X-(K/R)-X-(E/D)-(L/I/V/F) or (K/R)-X-X-X-(K/R)-X-(L/I/V/F), even more suitably the sequences (K/R)-X-X-(K/R)-X-(E/D)-(L/I/V/F)-X-(L/I/V/F), (K/R)-X-X-X-(K/R)-X-(L/I/V/F)-X-(L/I/V/F) or (K/R)-X-X-X-(K/R)-X-(E/D)-(L/I/V/F)-(L/I/V/F) and most suitably the sequence GRILARGEINFK (SEQ ID NO: 23).

SEQ ID NO: 23 is a sequence of the NCAM Ig2 domain and it therefore referred to as Ig2-peptide, Ig2-P or simply P2.

In another embodiment the compound may comprise the identified Ig2-peptide demonstrated to have the sequence GRILARGEINFK (SEQ ID NO:23) and not sharing any similarity to other neuritogenic peptides, either derived from the entire NCAM-sequence or found to bind the NCAM-molecule.

In a further embodiment the invention relates to compounds which bind to the part of the homophilic binding site of NCAM Ig1-Ig2 constituted by the Ig2 domain. Such peptides appear to have the general sequence, including any functional derivative thereof, as follows:
(Xaa)_{q}(Xaa⁻)-(Xaa)-(Xaa)-(Xaa)ₘ(Xaa)ₙ-(Xaa⁻)-(Xaa)-(Xaa⁺)-(Xaa^{h})-(Xaa^{h})ₚ-(Xaa^{h}),
wherein Xaa⁺ is a basic amino acid residue,
Xaa⁻ is a an acidic amino acid residue,
Xaa^{h} is an apolar amino acid residue,
Xaa is any amino acid residue, and
m, n, o, p and q independently are 0 or 1,
   and wherein the basic amino acid residues preferably are lysine or arginine, the acidic amino acids preferably are glutamic acid or aspartic acid, the apolar amino acids are preferably leucine, isoleucine, valine or phenylalanine, and r preferably is

A peptide within group III comprises the sequence (E/D)-X-X-X-(E/D)-X-(K/R)-(L/I/V/F)-X-((L/I/V/F)-X-(L/I/V/F), wherein X is any amino acid residue, suitably the sequence (E/D)-X-(K/R)-(L/I/V/F)-X-(L/I/V/F), (E/D)-X-(K/R)-(L/I/V/F)-(L/I/V/F), (E/D)-X-X-X-X-(E/D)-X-(K/R)-(L/I/V/F), (E/D)-X-X-X-(E/D)-X-(K/R)-(L/I/V/F) or (E/D)-X-X-(E/D)-X-(K/R)-(L/I/V/F), more suitably E/D)-X-X-(E/D)-X-(K/R)-(L/I/V/F)-X-(K/R)-(L/I/V/F) or (E/D)-X-X-(E/D)-X-(K/R)-(L/I/V/F)-(L/I/V/F), even more suitably the sequences (E/D)-X-X-X-X-(E/D)-X-(K/R)-(L/I/V/F)-(L/I/V/F), (E/D-X-X-X-(E/D)-X-(K/R)-(L/I/V/F)-X-(L/I/V/F) or (E/D)-X-X-X-(E/D)-X-(K/R)-(L/I/V/F)-(L/I/V/F), and most suitably the sequence GEISVGESKFFL (SEQ ID NO: 24).

SEQ ID NO: 24 is a sequence of the NCAM Ig1 domain and it therefore referred to as Ig1-peptide, Ig1-P or simply P1.

In a further aspect, the present invention relates to compounds which are anti-NCAM Ig1 antibodies, or antibodies recognising the part of Ig2 contributing to the NCAM Ig1-Ig2 binding site disclosed herein.

The antibodies may be monoclonal or polyclonal. Recombinant antibodies such as chimeric and/or humanised antibodies are also a part of the invention.

Such other compound may also be an anti-NCAM Ig1 antibody, an ant-NCAM Ig2 antibody (monoclonal, polyclonal or recombinant) or another antibody recognising epitopes in or near the binding site, that is constituted by the NCAM Ig1 and Ig2 domains, which antibody further may be chimeric or humanised. The production of polyclonal as well as a) monoclonal anti-NCAM Ig1 antibodies and/or b) anti-NCAM Ig2 antibodies may follow common known procedures. Mice or rabbits may serve as the primary immunisation forum, in which antibodies to NCAM Ig1 or antibodies to NCAM Ig2 are raised. Purified polyclonal antibodies may be used without any further treatment. Alternatively, monoclonal antibodies may be produced. Methods of producing monoclonal antibodies are common in the art. Recombinant antibodies such as chimeric and humanised antibodies may also be obtained by methods common in the art. Possible active antibodies are then screened according to the methods disclosed above or in similar ways.

The present invention provides thus compounds or compositions comprising the sequences shown above or derivatives hereof, such as peptide-analogues, peptide-fragments, polypeptides comprising the sequence or analogues hereof and non-peptide molecules derived from the herein presented peptides which are capable of stimulating survival of neurons, neuronal cell lines -or tissues.

These mentioned compounds and compositions can be used to treat conditions affecting the peripheral and/or the central nervous system and/or muscles and other tissues expressing NCAM or NCAM ligands as well as other conditions in which a stimulation of NCAM function or the function of NCAM ligand is beneficial.

A variety of suitable fragments and variants of NCAM has been discussed above. To be able to identify candidate ligands capable of stimulating NCAM function, where the NCAM function means the function of the NCAM molecule, the inventors have established a simple cell culture system (aggregate cell cultures) that permits a quantitative evaluation of the effect of various ligands. Hippocampal cells are provided from rat embryos. The cells are grown in a defined medium and dissociated cells are seeded in microtiter plates. After 24 h, the, amount of aggregates are counted. Compounds to be tested are added to the cell suspension immediately before seeding of the cells in micro-wells. When NCAM Ig1 binding ligands are present during the aggregation of cells, smaller, but more numerous cell aggregates are seen when quantified 24 h after seeding of the cells. The inhibiting effect of the ligands results in a blockage of the formation of large aggregates from many small aggregates as the adhesion, properties of NCAM are blocked. Thus, small but more numerous cell aggregates are seen in the presence of active ligands.

Such an effect was observed when different ligands of the NCAM Ig1-Ig2 domain were present during the aggregation of cells. Thus, the entire recombinant Ig1, and respectively Ig2, and a synthetic peptide derived from either the Ig2 sequence (Ig2-p) (SEQ ID NO:23) or a synthetic peptide derived from either the Ig1 sequence (Ig1-p) (SEQ ID NO:24) and peptide ligands of NCAM-Ig1 identified from libraries of synthetic peptides (SEQ ID NO:1, SEQ ID NO:2 and SEQ ID NO:3) inhibited aggregation in the described cell culture system.

The system allows the examination of disaggregation of the treated cells.

Putative artificial ligands may be selected and identified from peptide or non-peptide libraries. Any peptide library may be used. Synthetic peptide libraries as well as libraries containing fragmented natural occurring proteins, may be used in the search for useful peptides. Any kind of libraries comprising non-peptide compounds may similarly be used.

Peptides characterised by a certain sequence of amino acids may mimic a certain area of a protein. Naturally occurring proteins consist of L-amino acid residues. However, artificial peptides may also consist of or comprise D-amino acid residues. By combinatorial chemistry, mixtures of beads carrying peptides of equal length can be constructed, in which each bead carries peptides of a unique sequence (Lam et al., 1991). Such a mixture of peptides on beads is called a peptide library.

In the present invention, peptides were identified by screening synthetic random peptide libraries comprising resin-bound peptides with purified recombinant NCAM Ig1. The synthesis of the resin-bound one-bead one-peptide library was performed using the portioning, mix procedure (Furka, À., Sebestyyén, F., Asgedom, M. And Dibó, G. (1991) Int. J. Pep. Prot. Res. 37, 487493). Polyethylene syringes served as reaction vessels throughout the synthesis. Screenings were done by incubating the resin with biotinylated NCAM Ig1. Subsequently the resin was incubated with avidin-alkaline phosphatase. The substrates BCIP/NBT (Sigma) were added as described by Lam et al. (1992) and stained beads removed for micro sequencing.

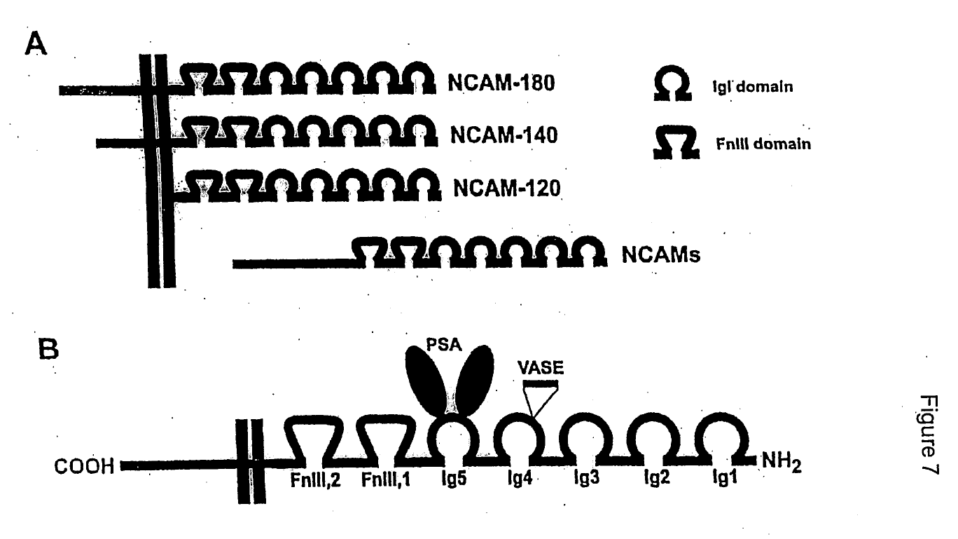

The most intensely stained beads were selected under stereo microscope and sequenced on an ABI 470A equipped with an ABI 120A HPLC. 22 NCAM Ig1 binding peptide sequences were identified (FIG. 7(A); SEQ ID NO:1 to SEQ ID NO:22).

It is to be understood that the method chosen for identification and selection of interesting peptides is not critical for the identification of a putative motif.

Libraries of small organic compounds may be screened to identify artificial ligands of the domains of NCAM. Further, screening for artificial ligands of the NCAM Ig1 and Ig2 domain, specifically the NCAM Ig2 domain Ig1-Ig2 binding site, that is constituted by the NCAM Ig1 and Ig2 domains. Such libraries or their construction are commonly known and the screening for useful ligands may follow the methods for screening disclosed in the present specification, or in ways obvious to the skilled person.

In a further aspect, the present invention relates to the polypeptides, fragments thereof or variants thereof, mimics thereof for use in the stimulation of survival of NCAM presenting cells and/or NCAM ligand presenting cells. The treatment is a treatment for preventing diseases and conditions of the central and peripheral nervous system, of the muscles or of various organs.

As discussed above, the present invention relates to treatment of individuals for preventing cell death of NCAM presenting cells or NCAM ligands presenting cells in vitro or in vivo, the treatment involving administering an effective amount of one or more compounds as defined above.

Treatment by the use of the compounds according to the invention is useful for stimulation of survival of cells which are at risk of dying due to a variety of factors, such as traumas and injuries, acute diseases, chronic diseases and/or disorders, in particular degenerative diseases normally leading to cell death, other external factors, such as medical and/or surgical treatments and/or diagnostic methods that may cause formation of free radicals or otherwise have cytotoxic effects, such as X-rays and chemotherapy. In relation to chemotherapy the NCAM binding compounds according to the invention are useful in cancer treatment since not all cancer cells express NCAM.

Also, the compounds according to the invention may be used for stimulating survival of cells being implanted or transplanted. This is particularly useful when using compounds having a long term effect, such as C3 discussed above.

In another aspect of the invention the compounds may be synthesised and secreted from implanted or injected gene manipulated cells.

Thus, the treatment comprises treatment and/or prophylaxis of cell death in relation to diseases or conditions of the central and peripheral nervous system, such as postoperative nerve damage, traumatic nerve damage, e.g. resulting from spinal cord injury, impaired myelination of nerve fibers, postischaemic damage, e.g. resulting from a stroke, multiinfarct dementia, multiple sclerosis, nerve degeneration associated with diabetes mellitus, neuro-muscular degeneration, schizophrenia, Alzheimer's disease, Parkinson's disease, or Huntington's disease.

Also, in relation to diseases or conditions of the muscles including conditions with impaired function of neuro-muscular connections, such as genetic or traumatic atrophic muscle disorders; or for the treatment of diseases or conditions of various organs, such as degenerative conditions of the gonads, of the pancreas, such as diabetes mellitus type I and II, of the kidney, such as nephrosis the compounds according to the invention may be used for preventing cell death, i.e. stimulating survival.

Furthermore, the compound is for the stimulation of the survival of heart muscle cells, such as survival after acute myocardial infarction.

Another aspect of the invention is the use of the compounds according to the invention in combination with a prosthetic nerve guide.

The compounds used according to the invention is preferably an oligomer (multimer) of monomers, wherein each monomer is as defined for the compound above. Particularly, multimeric peptides such as dendrimers may form conformational determinants or clusters due to the presence of multiple flexible peptide monomers. In one embodiment the compound is a dimer. In another embodiment the compound is a dendrimer, such as four peptides linked to a lysine backbone, or coupled to a polymer carrier, for example a protein carrier, such as BSA. The compound preferably comprises monomers independently capable of stimulating survival of cells presenting NCAM or an NCAM ligand/counterreceptor.

The individual monomers may be homologous, i.e. identical to one another, or the individual monomers may be heterologous, i.e. different from one another. The latter type of monomers may comprise at least two different monomers. In general dimers and multimers may comprise two or more identical monomers, or two or more monomers different from one another.

The invention also relates to a pharmaceutical composition comprising one or more of the compounds as defined above. In a preferred embodiment, the peptides are formulated as multimers, e.g. bound to carriers. The peptides may suitably be formulated as dendrimers such as four peptides linked to a lysine backbone, or coupled to a polymer carrier, for example a protein carrier, such as BSA Such formulations are well-known to the person skilled in the art.

In the present context the term pharmaceutical composition is used synonymously with the term medicament, therapeutic agent, pharmaceutical agent or drug which refers to any therapeutic or prophylactic agent which may be used in the treatment (including the prevention, diagnosis, alleviation, or cure) of a malady, affliction, condition, disease or injury in a patient. Therapeutically useful genetic determinants, peptides, polypeptides, nucleotides and polynucleotides, including derivatives thereof, is included within the meaning of the term pharmaceutical agent or drug. As defined herein, a “therapeutic agent,” “pharmaceutical agent” or “drug” or “medicament” is a type of bioactive agent. A bioactive agent is any substance or agent which may be used in connection with an application that is therapeutic or diagnostic, such as, for example, in methods for diagnosing the presence or absence of a disease in a patient and/or methods for the treatment of a disease in a patient. “Bioactive agent” refers to substances which are capable of exerting a biological effect in vitro and/or in vivo. The bioactive agents may be neutral, positively charged or negatively charged. Suitable bioactive agents include, for example, prodrugs, diagnostic agents, therapeutic agents, pharmaceutical agents, drugs, oxygen delivery agents, blood substitutes, synthetic organic molecules, proteins, peptides, vitamins, steroids, steroid analogs and genetic determinants, including nucleosides, nucleotides and polynucleotides.

The scope of the invention further concerns use of a method of preventing death of cells in vitro or in vivo, wherein the method involves administering, in vitro or in vivo an effective amount of one or more of the compounds described above or a composition as described below, so as to provide a stimulation of survival of NCAM presenting cells and/or NCAM ligand presenting cells in several tissues and organs as discussed herein. The medicament of the invention comprises an effective amount of one or more of the compounds as defined above, or a composition as defined above in combination with pharmaceutically acceptable additives. Such medicament may suitably be formulated for oral, percutaneous, intramuscular, intravenous, intracranial, intrathecal, intracerebroventricular, intranasal or pulmonal administration.

For most indications a localised or substantially localised application is preferred. The compounds are in particular used in combination with a prosthetic device such as a prosthetic nerve guide. Thus, in a further aspect, the present invention relates to a prosthetic nerve guide, characterised in that it comprises one or more of the compounds or the composition defined above. Nerve guides are known in the art.

Strategies in formulation development of medicaments and compositions based on the compounds of the present invention generally correspond to formulation strategies for any other protein-based drug product. Potential problems and the guidance required to overcome these problems are dealt with in several textbooks, e.g. “Therapeutic Peptides and Protein Formulation. Processing and Delivery Systems”, Ed. A. K. Banga, Technomic Publishing A G, Basel, 1995.

Injectables are usually prepared either as liquid solutions or suspensions, solid forms suitable for solution in, or suspension in, liquid prior to injection. The preparation may also be emulsified. The active ingredient is often mixed with excipients which are pharmaceutically acceptable and compatible with the active ingredient. Suitable excipients are, for example, water, saline, dextrose, glycerol, ethanol or the like, and combinations thereof. In addition, if desired, the preparation may contain minor amounts of auxiliary substances such as wetting or emulsifying agents, pH buffering agents, or which enhance the effectiveness or transportation of the preparation.

Formulations of the compounds of the invention can be prepared by techniques known to the person skilled in the art. The formulations may contain pharmaceutically acceptable carriers and excipients including microspheres, liposomes, microcapsules, nanoparticles or the like.

The preparation may suitably be administered by injection, optionally at the site, where the active ingredient is to exert its effect. Additional formulations which are suitable for other modes of administration include suppositories, nasal, pulmonal and, in some cases, oral formulations. For suppositories, traditional binders and carriers include polyalkylene glycols or triglycerides. Such suppositories may be formed from mixtures containing the active ingredient(s) in the range of from 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and generally contain 10-95% of the active ingredient(s), preferably 25-70%.

Other formulations are such suitable for nasal and pulmonal administration, e.g. inhalators and aerosols.

The active compound may be formulated as neutral or salt forms. Pharmaceutically acceptable salts include acid addition salts (formed with the free amino groups of the peptide compound) and which are formed with inorganic acids such as, for example, hydrochloric or phosphoric acids, or such organic acids as acetic acid, oxalic acid, tartaric acid, mandelic acid, and the like. Salts formed with the free carboxyl group may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The preparations are administered in a manner compatible with the dosage formulation, and in such amount as will be therapeutically effective. The quantity to be administered depends on the subject to be treated, including, e.g. the weight and age of the subject, the disease to be treated and the stage of disease. Suitable dosage ranges are of the order of several hundred μg active ingredient per administration with a preferred range of from about 0.1 μg to 1000 μg, such as in the range of from about 1 μg to 300 μg, and especially in the range of from about 10 μg to 50 μg. Administration may be performed once or may be followed by subsequent administrations. The dosage will also depend on the route of administration and will vary with the age and weight of the subject to be treated. A preferred dosis would be in the interval 30 mg to 70 mg per 70 kg body weight.

Some of the compounds of the present invention are sufficiently active, but for some of the others, the effect will be enhanced if the preparation further comprises pharmaceutically acceptable additives and/or carriers. Such additives and carriers will be known in the art. In some cases, it will be advantageous to include a compound, which promote delivery of the active substance to its target.

In many instances, it will be necessary to administrate the formulation multiple times. Administration may be a continuous infusion, such as intraventricular infusion or administration in more doses such as more times a day, daily, more times a week, weekly, etc. It is preferred that administration of the medicament is initiated before or shortly after the individual has been subjected to the factor(s) that may lead to cell death. Preferably the medicament is administered within 8 hours from the factor onset, such as within 5 hours from the factor onset. Many of the compounds exhibit a long term effect whereby administration of the compounds may be conducted with long intervals, such as 1 week or 2 weeks.

In connection with the use in nerve guides, the administration may be continuous or in small portions based upon controlled release of the active compound(s). Furthermore, precursors may be used to control the rate of release and/or site of release. Other kinds of implants and well as oral administration may similarly be based upon controlled release and/or the use of precursors.

The following are non-limiting examples illustrating the present invention.

### EXAMPLE 1

The Ig1 domain of NCAM was produced as a recombinant protein in Pichia pastoris. The cDNA fragment of rat NCAM was synthesised by PCR, and amplified cDNA was subcloned into an Xho I/Bam HI site of the pHIL-S1 plasmid (Invitrogen Corporation, San Diego, USA). An E. coli strain Top 10 F′ (Invitrogen Corporation, San Diego, USA) was used for transformation. The recombinant plasmid was linearised with Nsi I and used for transformation of Pichia pastoris strain His 4 GS-115 (Invitrogen Corporation, San Diego, USA). Transformation and selection was performed according to a Pichia Expression Kit manual supplied by the manufacturer. The recombinant protein was designated as Ig1 PP (Ig-like domain 1 produced in P. pastoris) The authenticity of Ig1 PP was secured by amino acid sequencing and MALDI-MS confirming the expected molecular weight of 11 kD. Cells were grown essentially according to the Pichia Expression Kit manual. After induction supernatant from growing cells was filtered through a 0.21 mm filter, concentrated by ultrafiltration and purified by gel filtration using a Sephadex G-50 column (Pharmacia Biotech AB, Sweden) (Thomsen et al., 1996).

### EXAMPLE 2

The cDNA encoding the Ig2 domain of NCAM was synthesised by PCR. Rat NCAM-120 cDNA was used as template. The amplified cDNA fragment was subcloned into a SnaBl/AvrII site of the pPIC9K plasmid (Invitrogen). The recombinant plasmid was linearised with SacI and used for transformation of Pichia pastoris strain His 4 GS-115 (Invitrogen) according to the protocol supplied by the manufacturer. The recombinant Ig2 domain of NCAM was expressed after induction in a 2 litre fermentor (MBR Mini Bioreactor, MBR Bioreactor AG). Thereafter, the expression medium was concentrated 10 times by ultra-filtration. The Ig2 domain was purified by gel-filtration by means of Sephadex G25 (Pharmacia) followed by ion exchange chromatography using a 5 ml HiTrap SP column (Pharmacia) yielding 10-15 mg. per litre of expression medium. The authenticity of the NCAM Ig2 domain was confirmed by amino acid sequencing and mass spectroscopy. In the N-terminal the original residues Lys-1 and Leu-2 were replaced with Tyr-1 and Val-2 due to cloning site considerations (Jensen et al., 1999).

The disclosed model of dimerization of the first two domains of NCAM was experimentally demonstrated by the use of a group mutation approach as follows.

Three mutants of NCAM (20-208) were produced. In the first mutant residues Glu-30, Glu-35 and Lys-37 from the homophilic binding site of domain-1 were substituted with Ala. In the second mutant residues Arg-192, Arg-196 and Glu-198 from the homophilic binding site of domain 2 were substituted with Ala. The third mutant had 6 residues Glu-30, Glu-35, Lys-37, Arg-192, Arg-196 and Glu-198 substituted with Ala.

Following the confirmation of the presence of mutations by restriction analysis and DNA sequencing, it was verified that there were no significant variations in expression levels or in purification patterns for the mutants in comparison with the unmutated NCAM(20-208). By the use of gel filtration chromatography it was revealed that NCAM(20-208) elutes as a dimer at ˜46 kDa, which finding provides for offering an easy and reliable way of monitoring the effects caused by mutations in the homophilic binding site when compared to the finding that the mutated proteins appeared to elute as monomers at ˜23 kDa. Thus, it was demonstrated that the mutations abolish the dimer formation suggesting that one or several pairs of the six mutated residues are involved in the dimer formation.

It was shown by the use of ¹H NMR spectra of each of the three mutated proteins that both domain-1 and domain-2 of the mutated double domains are folded very similarly into folds in the unmodified proteins (Jensen et al., 1999).

### EXAMPLE 3

The synthesis of the resin-bound one-bead one-peptide library was performed using the portioning, mix procedure (Furka et al., 1991). Polyethylene syringes served as reaction vessels throughout the synthesis and the final TFA-deprotection. TentaGel resin (Rapp Polymere, Tübingen, Germany) was divided into 18 aliquots and the protein L-amino acids except cysteine and histidine were used. Side-chains were protected with the following protecting groups: Asp(tBu), Glu(tBu), Tyr(tBu), Ser(tBu), Thr(tBu), Asn(trt), Gln(trt), Lys(Boc), Trp(Boc), Arg(pmc). Fmoc-protected amino acids (5 eq; Milligen or Novabiochem) were coupled overnight using 5eq DIC and 5eq HOBt. Removal of the Fmoc group was accomplished with 25% piperidine in DMF for 20 min. The side chain protecting groups were removed with 82.5% TFA, 5% anisole, 5% H₂O, 5% EDT, 2.5% thioanisole at mom temperature for 2.5 h followed by washing with tetrahydrofuran and 1% HOAc and the resin was subsequently lyophilised. Screenings were done by incubating 2 ml resin (equivalent to ca. 10⁶ beads) with biotinylated receptor in Tris/HCl buffer (Tris/HCl 0.025 M, pH 7.2, 0.25 M NaCl, 0.1 % (w/v) Tween 20) containing 0.1% Gelatin (Sigma) for 60 min. Subsequently the resin was washed in Tris/HCl buffer and incubated with avidin-alkaline phosphatase (diluted 1:20000) for 30 min. The substrates BCIP/NBT (Sigma) were added as described by Lam et al. (1992) and stained beads were removed for micro sequencing. The library was screened with the receptor NCAM Ig1-PP (10 mg/ml).

### EXAMPLE 4

The most intensely stained beads were selected under stereo microscope and sequenced on an ABI 470A equipped with an ABI 120A HPLC. The 2 peptide sequences obtained (SEQ ID NO:2 to SEQ ID NO:3). Peptide sequences to be synthesised and used in further investigations were chosen by aligning the obtained sequences and examining these for repeated patterns revealing putative motifs.

### EXAMPLE 5

One peptide, P1 (SEQ ID NO: 24) derived from the sequence of NCAM Ig1 was synthesised as, described below. In addition, one peptide, P2 (SEQ ID NO: 23) derived from the sequence of NCAM Ig2 was synthesised as described below. From combinatorial libraries 22 NCAM Ig1-binding sequences (SEQ ID NO: 1-22) were identified.

Three peptides, C3 (SEQ ID NO: 1), D3 (SEQ ID NO: 2) and D4 (SEQ ID NO:3) were selected for further analysis and synthesised on TentaGel resin with Rink amide linker (p-((R,S)-α-(I-(9H-fluoren-9-yl)-methoxyformamido)-2,4-dimethoxybenzyl)-phenoxy-acetic acid (Novabiochem)) using Fmoc-protected amino acids (3 eq.). Coupling was performed for >60 min. with TBTU (3 eq.), HOBt (3 eq.) and DIEA (4.5 eq.) in a manual multicolumn apparatus. Fmoc was deprotected with 20% piperidine in DMF for 10 min. Synthesis of peptide dendrimers was accomplished by coupling Fmoc-Lys(Fmoc)-OH (Novabiochem) to the linker resin followed by Fmoc-deprotection of the Fmoc group and further coupling of Fmoc-Lys(Fmoc)-OH was performed. After Fmoc-deprotection the synthesis of peptides was performed as above for the monomeric peptides. Peptidyl resins were deprotected with TFA 90%, 5% H₂O, 3% EDT, 2% thioanisole, precipitated in diethyl ether, washed three times in diethyl ether, solubilised in 5% AcOH and lyophilised. Amino acid analysis was performed using Waters picotag and Waters 501 pump connected to WISP 712. Waters 600E equipped with Waters 996 photodiode array detector was used for analytical and preparative HPLC on C₁₈ columns (Delta-Pak 100 Å15 um, Millipore) MALDI-MS was done on a VG TOF Spec E, Fisions Instrument. The peptides were at least 95% pure as estimated by HPLC.

### EXAMPLE 6

Real-time biomolecular interaction analysis was performed using a BIAlite instrument (Pharmacia Biosensor AB, Sweden). All experiments were performed at 25° C. using Hepes buffered saline (HBS: 10 mM Hepes pH 7.4, 150 mM NaCl, 3.4 mM EDTA, 0.005% v/v Surfactant P20 (Pharmacia Biosensor, Sweden) as running buffer. The flow rate was 5 ml/min. Dendrimer peptides C3d, D3d and D4d (four peptide-monomers coupled to a backbone consisting of three lysines) were immobilised on a sensor chip CM5 (Pharmacia Biosensor AB, Sweden) using the following procedure: the chip was activated by 10 ml 0.05 M N-hydroxysuccinimide, 0.2 M N-ethyl-N′-(dimethylaminopropyl)carbodiimide; peptides were immobilised using 35 ml peptide solution in HBS at a concentration of 60 μg/ml; finally the chip was blocked by 35 μl 1 M ethanolamine hydrochloride pH 8.5. Binding of Ig1 to dendrimer peptides: 50 ml of Ig1 or Ig1I at the indicated concentrations were applied. The chip was regenerated by two 5 ml pulses of 5 mM NaOH. Two independent experiments were performed. The results confirmed that C3d, D3d and D4d bind to the NCAM Ig1 domain.

The invention is further illustrated by the following non-limiting examples.

### EXAMPLE 7

Four different experimental paradigms have been used to test the effect of C3d on the survival of neurons.

a) (NGF Withdrawal)

In the first experimental set-up, a subclone of PC12 cells (PC12-E2) was employed. Pheochromocytoma (PC12) cell line is an established model of neuronal cell-line, which can be differentiated into a sympathetic-type of neurons by treatment with Nerve Growth Factor (NGF). This cell line has been widely used in neurobiological studies.

PC12 cells were differentiated by plating them in 96-well tissue culture plates in defined medium (DMEM) supplemented with NGF (50-100 ng/ml) and were normally used after 6-8 days of NGF treatment. NGF withdrawal was performed as follows: the medium was removed, and the cells were quickly rinsed twice with pre-warmed medium, and then re-fed with DMEM supplemented with C3d. After 2 days of incubation cells were assayed for cell survival by measuring reduction of a novel tetrazolium compound, MTS (Promega, USA), which is bioreduced by cells into a formazan that is soluble in tissue culture medium. The absorbance of the formazan at 490 nm was measured directly from 96 well assay plates without additional processing. The conversion of MTS into the aqueous soluble formazan was accomplished by dehydrogenase enzymes found in metabolically active cells. The quantity of formazan product as measured by the amount of 490 nm absorbance was directly proportional to the number of living cells in culture (Yao and Cooper, 1995; CellTiter 96 Aqueous non-radioactive cell proliferation assay, Promega, 1996; Eilers et al., 1998).

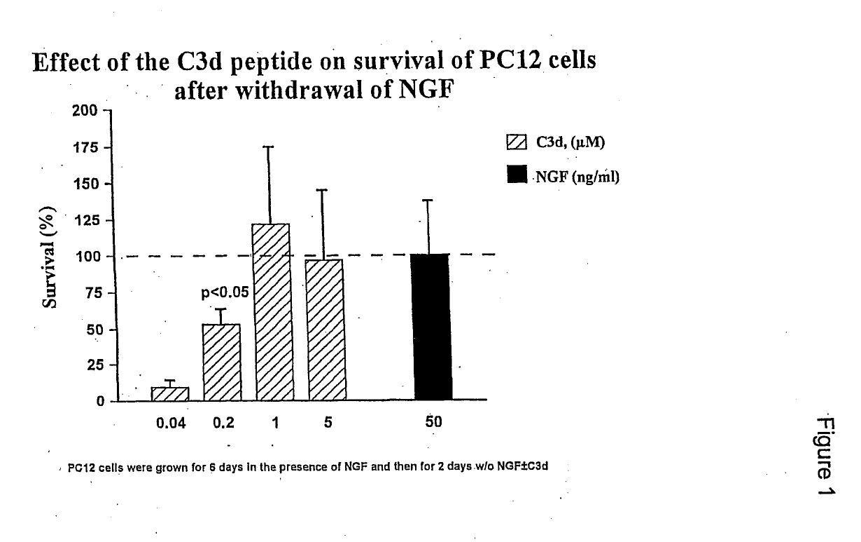

The result of survival according to the assay is shown in FIG. 1.

b) (Serum Deprivation)

In the second experimental paradigm PC12 cells were starved and the protective effect of C3d was analysed.

PC12 cells were plated in 96-well tissue culture plates in the absence of growth factors (serum starvation) for 6 days in DMEM supplemented with C3d and assayed for cell survival by reduction of MTS, as described above (Gollapudi and Oblinger, 1999; Williams and Doherty, 1999).

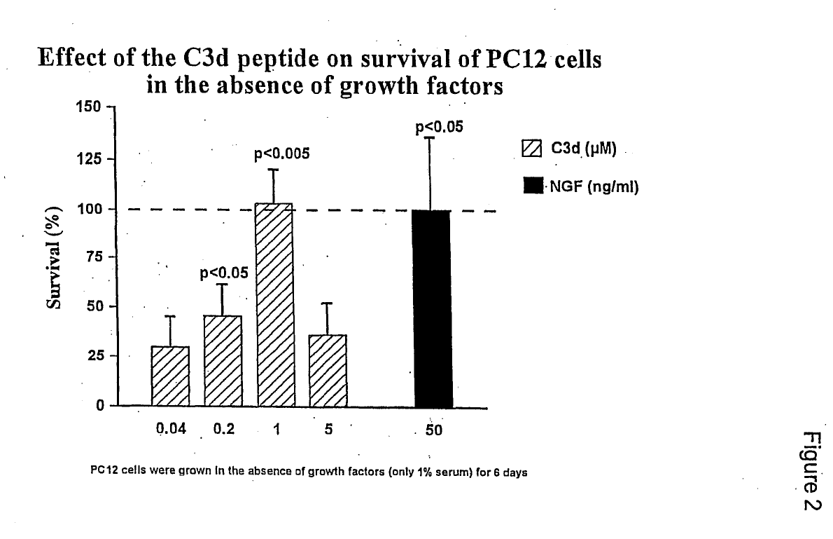

The result of survival according to the assay is shown in FIG. 2.

c) (Potassium Depolarisation)

In the third experimental paradigm primary cultures of rat cerebellar granule neurons (CGN) were employed. Cultured CGN die by apoptosis when switched from a medium containing an elevated (high) level of potassium (HK) to one with lower K⁺ (LK). When neurons grow in HK which depolarise cells, they differentiate. Death resulting from the lowering K⁺ can be prevented by e.g. adding brain-derived growth factor (BDNF) or other substances, which have a putative protective effect.

Cerebellar granule neurons (CGN) from 7-days old rats are grown for 7-8 days in the presence of HK (40 mM). Cells are washed twice with serum-free culture medium (basal Eagle's medium BME) containing LP (5 mM) and grown in serum-free medium supplemented with C3d for two days. Cultures are assayed for cell survival by measuring reduction of MTS, as described above. (D'Mello et al., 1997; Villalba et al., 1997; Skaper et al., 1998).

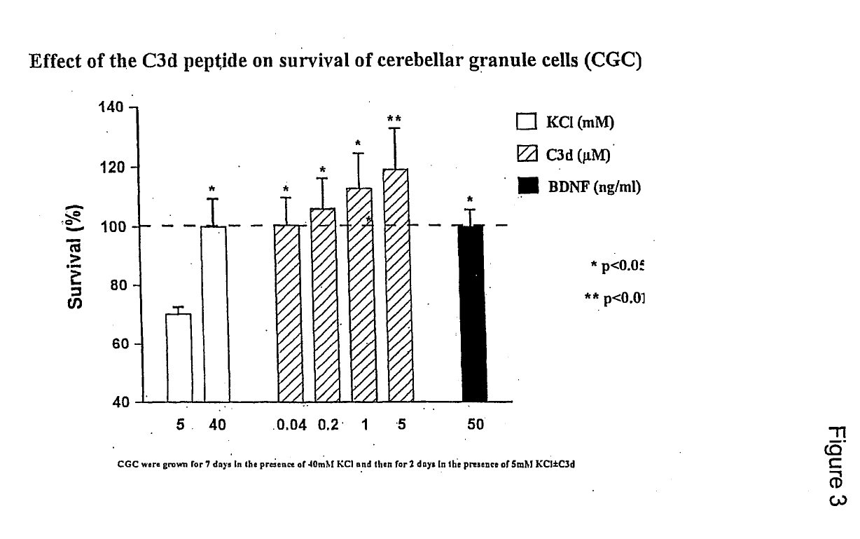

The result of survival according to the assay is shown in FIG. 3.

d) Survival of Dopaminergic Neurons Treated with 6-OHDA

In the fourth experimental paradigm, primary cultures of rat dopaminergic neurons (DN) from midbrain of 14 days old embryos were employed. After six days in vitro, DN treated with 6-OHDA for two hours die in a few days.

DN are grown for 6 days without 6-OHDA. Cells are washed twice with serum-free culture medium and grown for 2 hours in the presence of 100 μM 6-OHDA and the C3d peptides. After that medium containing 6-OHDA is removed and DN are grown for 2 days in the presence of C3d. Cultures are assays for cell survival by counting the number of neurons immunostained for the expression of tyrosinehydroxilase (Hulley P, Schachner M, Lubbert H. L1 neural cell adhesion molecule is a survival factor for fetal dopaminergic neurons. J Neurosci Res. 1998, 53:129-34).

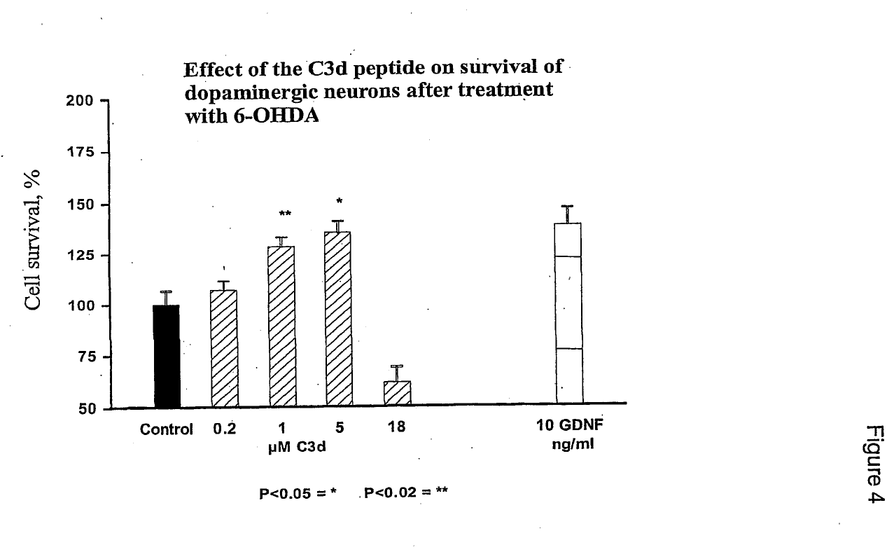

The result of survival according to the assay is shown in FIG. 4.

### EXAMPLE 8

Two different experimental paradigms were used to test the effect of the FGL peptide on the survival of neurons.

a) (Potassium Depolarization)

This was done as described above for C3d (for details see Example 7, c.)

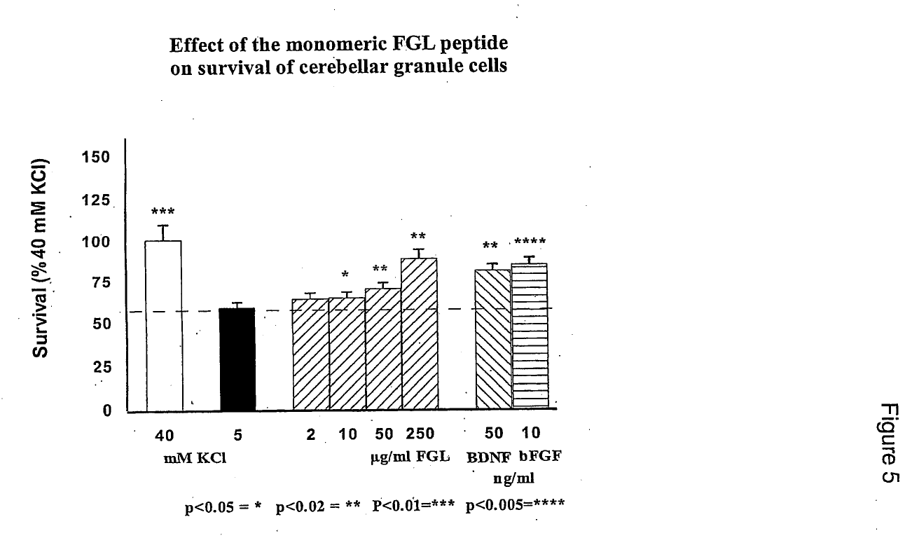

The result of survival according to the assay is shown in FIG. 5.

b) (Glutamate Excitotoxicity)

In the fourth experimental paradigm primary cultures of rat cerebellar granule neurons (CGN) were employed. Cultured CGN die by apoptosis when subjected to treatment with high concentration of glutamate (glutamate excitotoxicity). When neurons grow in medium containing high levels of potassium (HK), HK depolarizes cells causing their differentiation. Treatment of neurons with glutamate results in cell death (Brecht S. et al., 2001).

This was done as described above for C3d with one exception: instead of changing to the LK-medium after incubation of CGN in HK-medium, the glutamate (1 mM)-containing medium was used.

The result of survival according to the assay is shown in FIG. 9.

### REFERENCES

Andersson, A M., Biochemical Journal 1993; 290:641-8.
Berezin, V. et al., Curr. Opin. Drug Discov. Dev., 2000, 3, pp. 605-60
Beggs, H. E. N., Journal of Biological Chemistry 1997, 272, no. 13: 8310-8319.
(Brecht S, Gelderblom M, Srinivasan A, Mielke K, Dityateva G, Herdegen T., Brain
Res. 2001, 94:25-34).
Carenini, S., Cell & Tissue Research 1997; 287:3-9.
CellTiter 96 Aqueous non-radioactive cell proliferation assay, Promega, 1996.
Cremer, H., Molecular & Cellular Neurosciences 1997; 8:323-35.
Cremer, H., Nature 1994; 367:455-9.
Daniloff, J K., Journal of Cell Biology 1986; 103:929-45.
Daston, M M., Journal of Neuroscience 1996; 16:5488-97.
D'Mello, S. R., Borodezt K. and Soltoff, S. P., (1997) Insulin-like growth factor and potassium depolarization maintain neuronal survival by distinct pathways: possible involvement of PI 3-kinase in IGF-1 signaling. J. Neurosci. 17:1548-1560.
Doherty, P., Nature 1992; 356:791-3.
Doherty, P., Molecular and Cellular Neuroscience 1996; 8:99-111.
Doyle, E., Journal of Neuroscience Research 1992; 31:513-23.
Edelman, G M., Cold Spring Harbor Symposia on Quantitative Biology. Cold Spring
Eilers, A., Whitfield, J., Babij, C., Rubin, L. L and Ham J. (1998) Role of the Jun kinase pathway in the regulation of c-Jun expression and apoptosis in sympathetic neurons. J. Neurosci. 18:1713-1724.
Harbor Laboratory Press, 1990: 303-18.
Fazeli, S., Seminars in the Neurosciences 1997; 8:367-77.
Fields R D. Trends in Neurosciences 1996; 19:473-80.
Frei, T., Journal of Cell Biology 1992; 118:177-94.
Furka, A., International Journal of Peptide.& Protein Research 1991; 37: 487493.
Gaardsvoll, H., European Journal of Cell Biology 1993; 61:100-7.
Gollapudi, L. and Oblinger, M. M. (1999) Stable transfection of PC12 cells with estrogen receptor (ERalpha): Protective effects of estrogen on cell survival after serum deprivation. J. Neurosci. Res. 56:99-108.
Horstkorte, R., The Journal of Cell Biology 1993; 121, no 6:1409-21.
Jensen, P. H., et al., Nature Struct. Biol., 1999
Jucker, M., Brain Research 1995; Molecular Brain Rese:149-56.
Kasper, C., Journal of Neuroscience Research 1996; 46:173-86.
Kiselyov, V., Journal of Biological Chemistry 1997, 272: 10125-10134.
Knittel, T., American Journal of Pathology 1996; 149:449-62.
Krushel, L A., Proceedings of the National Academy of Sciences of the United States of America 1998; 95:2592-6.
Lackie, P M., Development 1990; 110:933-47.
Lahrtz, F., Journal of Neuroscience Research 1997; 50:62-8.
Lam, K S., Nature 1991, 354: 82-84.
Lam, K S., Immunomethods 1992,1,11-15.
Landmesser,. L., Neurone 1990; 4:655-67.
Luthi, A., Nature 1994; 372:777-9.
Maar, T E., Journal of Neuroscience Research 1997, 47: 163-172.
Massaro, A R., Italian Journal of Neurological Sciences 1987; Suppl 6:85-8.
Moller, C J., Anatomy & Embryology 1991; 184:541-8.
Moller, C J., Molecular Endocrinology 1992; 6:1332-42.
Nieke, J., Differentiation 1985; 30:141-51.
Olsen, M., Int J Devl Neuroscience 1995; 13:97-104.
Ono, K., Neurone 1994; 13:595-609.
Pollerberg, G E., Developmental Biology 1993; 156(2):324-40.
Rabinowitz, J E., Proceedings of the National Academy of Sciences of the United States of America 1996; 93:6421-4.
Ranheim, T. S., Proceedings of the National Academy of Sciences of the United States of America 1996, 93: 4071-4075.
Rao, Y., Journal of Cell Biology 1992; 118:937-49.
Rao, Y., Journal of Biological Chemistry 1994; 269:27540-8.
Retzler, C., et al., J. Biol. Chem., 1996, 271, 27304-27310 Romanska H M, Journal of Pediatric Gastroenterology & Nutrition 1996; 22:351-8.
Rønn, L C., Brain Research 1995; 677:145-51.
Rønn, L C., Ph.D.thesis; The Protein Laboratory and The Division of Neurophysiology, University of Copenhagen 1997.
Rutishauser, U., Trends in Neurosciences 1996; 19:422-7.
Sandig, M., Journal of Biological Chemistry 1994; 269:14841-8.
Sanes, J R., Journal of Cell Biology 1986; 102:420-31.
Schmid, R-S., Journal of Neurobiology 1999; 38:542-558.
Scholey, A B., Neuroscience 1993; 55:499-509.
Schuch, U., Neurone 1989; 3:13-20.
Shen, H., Journal of Neuroscience 1997; 17:5221-9.
Skarpe, S. D., Floreani M., Negro A., Facci L and Giusti P. (1998) Neurotrophins rescue cerebellar granule neurons from oxidative stress-mediated apoptotic death: Selective involvement of phosphatidylinositol 3-kinase and the mitogen-activated protein kinase pathway. J. Neurochem. 70:1859-1868.
Stahlhut, M., Journal of Neuroscience Research 1997; 48:112-21.
Stork, O., European Journal of Neuroscience 1997; 9:1117-25.
Thomsen, N K., Nature Structural Biology 1996; 3:581-5.
Van, Kammen D P., Biological Psychiatry 1998; 43:680-6.
Villalba, M., Bockaert J. and Journot L. (1997) Pituitary adenylate cyclase-activating polypeptide (PACAP-38) protects cerebellar granule neurons from apoptosis by activating the mitogen-activated protein kinase (MAP kinase) pathway. J. Neurosci. 17:83-90.
Walsh, F S., Neuroscience Letters 1985; 59:738.
Williams, E.-J., and Doherty P. (1999) Evidence for and against a pivotal role of PI-3-kinase in a neuronal cell survival pathway. Mol. Cell. Neurosci. 13:272-280.
Yao, R., and Cooper, G. M., (1995) Requirement for phosphatidylinositol-3 kinase in the prevention of apoptosis by nerve growth factor. Science 267:2003-2006.
Zhang, H., Journal of Neuroscience 1992; 12:3107-14.

1. Method for preventing cell death of cells presenting: the neural cell adhesion molecule (NCAM) or an NCAM ligand, comprising administering an effective amount of medicament comprising a compound comprising a peptide comprising at least 5 contiguous amino acid residues from the amino acid sequence (SEQ ID NO:1) of the neural cell adhesion molecule (NCAM) a fragment thereof, or a variant thereof or a mimic thereof.

2. The method according to claim 1, wherein said compound binds to the NCAM Ig1 domain.

3. The method according to claim 1, wherein said compound binds to the NCAM Ig2 domain.

4. The method according to claim 1, wherein said compound binds to the homophilic Ig1 binding site of the Ig1-Ig2 domains.

5. The method according to claim 1, wherein said compound binds to the homophilic Ig2 binding site of the Ig1-Ig2 domains.

6. The method according to claim 1, wherein said compound is the NCAM Ig2 polypeptide, or a fragment, or a variant thereof, or a mimic thereof.

7. The method according to claim 1, wherein said compound is the NCAM Ig1 polypeptide, or a fragment, or a variant, or a mimic thereof.

8. The method according to claim 1, wherein the compound binds to the NCAM Ig1 domain through a binding motif which comprises at least 2 basic amino acid residues.

9. The method according to claim 2, wherein the compound is an anti-NCAM Ig1 antibody.

10. The method according claim 3, wherein the compound is an anti-NCAM Ig2 antibody.

11. The method according to claim 3, wherein the compound is an antibody against the Ig2 domain.

12. The method according to claim 11, wherein the antibody is monoclonal.

13. The method according to claim 12, wherein the antibody is chimeric or humanised.

14. The method according to claim 1, wherein the compound is a non-polypeptide molecule.

15. The method according to claim 14, wherein the compound mimicks the binding to the homophilic binding site of the Ig1 -Ig2 domains constituted by the Ig1 domain, or a fragment thereof, or a polypeptide mimic thereof.

16. The method according to claim 1, wherein the compound is a polypeptide.

17. The method according to claim 1, wherein the compound comprises the binding motif comprising at least 2 basic amino acid residues, and at least 1 apolar amino acid.

18. The method according to claim 1, wherein the compound comprises the binding motif comprising at least 2 acidic amino acid residues, and at least 1 apolar amino acid.

19. The method according to claim 8, the compound comprising at least 2 basic amino acid residues within a sequence of 10 amino acid residues.

20. The method of claim 8, comprising at least 2 basic amino acid residues within a sequence of 3 amino acid residues.

21. The method according to claim 6, wherein the compound being a polypeptide comprising the sequence:
(Xaa⁺)ₘ-(Xaa)ₚ-(Xaa⁺)-(Xaa¹)ᵣ-(Xaa⁺)-(Xaa)_{q}-(Xaa⁺)ₙ;
wherein Xaa⁺ is a basic amino acid residue, Xaa¹ is any amino acid residue,
Xaa is any amino acid residue, and m, n, p, q and r independently are 0 or 1.

22. The method according to claim 21, wherein the basic amino acid residues are lysine (K) or arginine (R).

23. The method according to claim 21, wherein r is 1.

24. The method according to claim 21, wherein Xaa¹ is proline (P) or glutamic acid (E).

25. The method according to claim 21, wherein at least one of m and n is 1.

26. The method according to claim 16, wherein the polypeptide comprises the sequence (Lys/Arg)₀₋₁-(Lys/Arg)-Xaa₁-(Lys/Arg).

27. The method according to claim 26, wherein the polypeptide has the sequence A-S-K-K-P-K-R-N-I-K-A (SEQ ID NO:1), A-K-K-E-R-Q-R-K-D-T-Q (SEQ ID NO:2), or A-R-A-L-N-W-G-A-K-P-K (SEQ ID NO:3).

28. The method according to claim 21, wherein one or more of the amino acid residues is modified, such as being acetylated.

29. The method according to claim 16, wherein the polypeptide is identical to a part of the NCAM Ig2 domain.

30. The method according to claim 16, wherein the polypeptide is a variant of the NCAM Ig2 domain, or of a fragment of the NCAM Ig2 domain.

31. The method according to claim 16, wherein the polypeptide binds to the NCAM Ig2 binding site on the NCAM Ig1 domain.

32. The method according to claim 16, wherein the polypeptide binds to a binding site on the NCAM Ig1 domain different from the NCAM Ig2 binding site.

33. The method according to claim 17, wherein the number of amino acid residues in the sequence of the binding motif are at the most 12 amino acid residues.

34. The method according to claim 17, wherein the number of amino acid residues in the sequence of the binding motif is at the most 8 amino acid residues.

35. The method according to claim 2, wherein the compound being a polypeptide comprising the sequence:
(Xaa)_{q}-(Xaa⁺)-(Xaa)-(Xaa)-(Xaa)ₘ-(Xaa⁺)-(Xaa)-(Xaa⁻)ₙ-(Xaa^{h})-(Xaa)ₒ-(Xaa^{h})ₚ-(Xaa⁺)
wherein Xaa⁺ is a basic amino acid residue,
Xaa⁻ is a an acidic amino acid residue,
Xaa^{h} is a apolar amino acid-residue,
Xaa is any amino acid residue, and
m, n, o, p and q independently are 0 or 1.

36. The method according to claim 35, wherein the basic amino acid residues are lysine (K) or arginine (R).

37. The method according to claim 35, wherein the acidic amino acids are glutamic acid (E) or aspartic acid (D), the apolar amino acids are leucine (L), isoleucine (I), valine (V) or phenylalanine (F), and r is 1.

38. The method according to claim 35, wherein the polypeptide comprises the sequence (K/R)-X-X-X-(K/R)-X-(E/D)-(L/I/V/F)-X-(L/I/V/F), wherein X is any amino acid residue.

39. The method according to claim 38, wherein the polypeptide has the sequence GRILARGEINFK (SEQ ID NO:23).

40. The method according to claim 35, wherein one or more of the amino acid residues is a modified amino acid residue.

41. The method according to claim 35, wherein the polypeptide is a mimic or a fragment of, or is identical to a part of the homophilic binding site of the NCAM Ig1-Ig2 domains which is constituted by the Ig2 domain.

42. The method according to claim 18, wherein the number of amino acid residues in the sequence of the binding motif is at the most 12 amino acid residues.

43. The method according to claim 18, wherein the number of amino acid residues in the sequence of the binding motif is within 9 amino acid residues.

44. The method according to claim 3, wherein the compound being polypeptide comprising the sequence:
(Xaa)_{q}-(Xaa⁻)-(Xaa)-(Xaa)-(Xaa)ₘ-(Xaa)ₙ-(Xaa⁻)-(Xaa)-(Xaa⁺)-(Xaa^{h})-(Xaa^{h})ₚ-(Xaa^{h}),
wherein Xaa⁺ is a basic amino acid residue,
Xaa⁻ is an acidic amino acid residue,
Xaa^{h} is a apolar amino acid residue,
Xaa is any amino acid residue, and
m, n, o, p and q independently are 0 or 1.

45. The method according to claim 44, wherein the basic amino acid residues are lysine (K) or arginine (R).

46. The method according to claim 44, wherein the the acidic amino acids are glutamic acid (E) or aspartic acid (D), the apolar amino acids are leucine (L), isoleucine (I), valine (V) or phenylalanine (F), and r is 1.

47. The method according to claim 44, wherein the polypeptide comprises the sequence (E/D)-X-X-X-(E/D)-X-(K/R)-(L/I/V/F)-X-(L/I/V/F), wherein X is any amino acid residue.

48. The method according to claim 47, wherein the polypeptide has the sequence GEISVGESKFFL (SEQ ID NO: 24).

49. The method according to claim 44, wherein one or more of the amino acid residues is a modified amino acid.

50. The method according to claim 44, wherein the compound is a mimic of a part of the homophilic binding site of the NCAM Ig1-Ig2 domains which is constituted by the Ig1 domain.

51. The method according to claim 1, wherein the compound is a dimer.

52. The method according to claim 1, wherein the compound is a multimer, such as a dendrimer.

53. A method of treating a disease or condition of the central and peripheral nervous system, which comprises preventing cell death of cells presenting the neural cell adhesion molecule (NCAM) or a NCAM ligard by the method of claim 1.

54. A method of treating a disease or condition of the muscles including conditions with impaired function of neuro-muscular connections, which comprises preventing cell death of cells presenting the neural cell adhesion molecule (NCAM) or a NCAM ligard by the method of claim 1.

55. A method of stimulating the survival of heart muscle cells, which comprises preventing cell death of cells presenting the neural cell adhesion molecule (NCAM) or a NCAM ligard by the method of claim 1.


SEQUENCE LISTING (24 sequences)

SEQ ID NO: 1 - protein, 11 aa, Homo sapiens
ASKKPKRNIKA

SEQ ID NO: 2 - protein, 11 aa, Homo sapiens
AKKERQRKDTQ

SEQ ID NO: 3 - protein, 11 aa, Homo sapiens
ARALNWGAKPK

SEQ ID NO: 4 - protein, 11 aa, Homo sapiens
AGSAVKLKKKA

SEQ ID NO: 5 - protein, 11 aa, Homo sapiens
AKYVLIPIRIS

SEQ ID NO: 6 - protein, 10 aa, Homo sapiens
ASTKRSMQGI

SEQ ID NO: 7 - protein, 12 aa, Homo sapiens
ARRAILMQTNAL

SEQ ID NO: 8 - protein, 11 aa, Homo sapiens
AYYLIVRVNRI

SEQ ID NO: 9 - protein, 11 aa, Homo sapiens
ATNKKTGRRPR

SEQ ID NO: 10 - protein, 11 aa, Homo sapiens
AKRNGPLINRI

SEQ ID NO: 11 - protein, 11 aa, Homo sapiens
AKRSVQKLDGQ

SEQ ID NO: 12 - protein, 11 aa, Homo sapiens
ARQKTMKPRRS

SEQ ID NO: 13 - protein, 10 aa, Homo sapiens
AGDYNPDLDR

SEQ ID NO: 14 - protein, 11 aa, Homo sapiens
ARKTRERKSKD

SEQ ID NO: 15 - protein, 11 aa, Homo sapiens
ASQAKRRKGPR

SEQ ID NO: 16 - protein, 11 aa, Homo sapiens
APKLDRMLTKK

SEQ ID NO: 17 - protein, 11 aa, Homo sapiens
AKKEKPNKPND

SEQ ID NO: 18 - protein, 11 aa, Homo sapiens
AQMGRQSIDRN

SEQ ID NO: 19 - protein, 11 aa, Homo sapiens
AEGGKKKKMRA

SEQ ID NO: 20 - protein, 11 aa, Homo sapiens
AKKKEQKQRNA

SEQ ID NO: 21 - protein, 11 aa, Homo sapiens
AKSRKGNSSLM

SEQ ID NO: 22 - protein, 11 aa, Homo sapiens
ARKSRDMTAIK

SEQ ID NO: 23 - protein, 12 aa, Homo sapiens
GRILARGEINFK

SEQ ID NO: 24 - protein, 12 aa, Homo sapiens
GEISVGESKFFL